(19) United States
(12) Patent Application Publication
(10) Pub. No.: US 2011/0190863 A1
(43) Pub. Date: Aug. 4, 2011

(54) Therapeutic Balloon with Systemic Drug Loss Protection and Controlled Particle Size Release

(75) Inventors: Tim Ostroot, Cokato, MN (US); Derek Sutermeister, Eden Prairie, MN (US); James Anderson, Fridley, MN (US)

(73) Assignee: BOSTON SCIENTIFIC SCIMED, INC., Maple Grove, MN (US)

(21) Appl. No.: 13/014,269

(22) Filed: Jan. 26, 2011

Related U.S. Application Data
(60) Provisional application No. 61/301,018, filed on Feb. 3, 2010.

(51) Int. Cl. A61M 25/10; A61F 2/84

(52) U.S. Cl. 623/1.11; 604/103.02

(57) ABSTRACT

Systemic drug loss protection devices are disclosed. The drug loss protection device includes a balloon and a drug particulate filter. The drug particulate filter has a plurality of openings and is configured to selectively permit transmission of drug particulates through the filter. Consequently, the size of the openings in the filter regulates transmission of the drug particulates. The drug particulate filter can also be used as an embolic filter.

### CROSS-REFERENCE TO RELATED APPLICATIONS

This Application is a non-provisional of application No. 61/301,018, filed Feb. 3, 2010, which is herein incorporated by reference.

### STATEMENT REGARDING FEDERALLY SPONSORED RESEARCH

Not Applicable.

### BACKGROUND

The use of drug coated intraluminal medical devices is generally known. Previously known drug coated devices release drug particulates into a lumen, for example a blood vessel. In particular, known drug eluting balloons release drug particulates in an uncontrolled fashion. Particulates of various sizes can flow into the blood stream without restraint. In some cases, the release of drugs and drug particulates into a bloodstream can lead to an undesirable and potentially severe reaction in the patient due to increased toxicity and systemic loss of the drug or excipient.

Thus, there remains a need for a medical device that prevents unregulated release of particulates into a body lumen.

The art referred to and/or described above is not intended to constitute an admission that any patent, publication or other information referred to herein is “prior art” with respect to this disclosure. In addition, this section should not be construed to mean that a search has been made or that no other pertinent information as defined in 37 C.F.R. §1.56(a) exists.

All US patents and applications and all other published documents mentioned anywhere in this application are incorporated herein by reference in their entirety.

Without limiting the scope of this disclosure a brief summary of some of the claimed embodiments is set forth below. Additional details of the summarized embodiments and/or additional embodiments of the may be found in the Detailed Description, below.

### BRIEF SUMMARY

In some embodiments, an intravascular drug loss protection device comprises an inflatable balloon and a drug particulate filter adjacent to the inflatable balloon. In some embodiments, the drug particulate filter comprises a mesh. In some embodiments, the mesh defines a plurality of openings configured to selectively capture drug particulates.

### BRIEF DESCRIPTION OF THE SEVERAL VIEWS OF THE DRAWING(S)

FIG. 1 shows a side view of an embodiment of the drug loss protection device with drug delivery system.

FIG. 2A shows a side view of the embodiment of the drug loss protection device of FIG. 1 in a delivery configuration.

FIG. 2B shows a side view of the embodiment of the drug loss protection device of FIG. 1 in an expanded configuration.

FIG. 2C shows a side view of the embodiment of the drug loss protection device of FIG. 1 in a post-expansion configuration.

FIG. 2D shows a side view of the embodiment of the drug loss protection device of FIG. 1 in an unexpanded configuration.

FIG. 3 shows a detailed view of the embodiment of the drug loss protection device of FIG. 1.

FIG. 3B shows a detailed view of an embodiment of the drug loss protection device.

FIG. 4 shows a detailed view of the embodiment of the drug loss protection device of FIG. 1.

FIG. 5 shows a side view of an embodiment of the drug loss protection device.

FIG. 6 shows a side view of an embodiment of the drug loss protection device.

FIG. 7 shows a side view of an embodiment of the drug loss protection device.

### DETAILED DESCRIPTION

While this invention may be embodied in many different forms, there are described in detail herein specific embodiments. This description is an exemplification of the principles of the invention and is not intended to limit the invention to the particular embodiments illustrated.

For the purposes of this disclosure, like reference numerals in the figures shall refer to like features unless otherwise indicated.

In at least one embodiment, a balloon catheter comprises a drug loss protection device having a balloon and a particulate filter. In some embodiments, the balloon has a drug disposed thereon. In some embodiments, the drug is applied to an inside of a body lumen, for example a blood vessel or artery. In some embodiments, drug particulates no larger than a predetermined size are permitted to exit the device. In this way, particulates larger than the predetermined size are prevented from flowing downstream of the drug loss protection device.

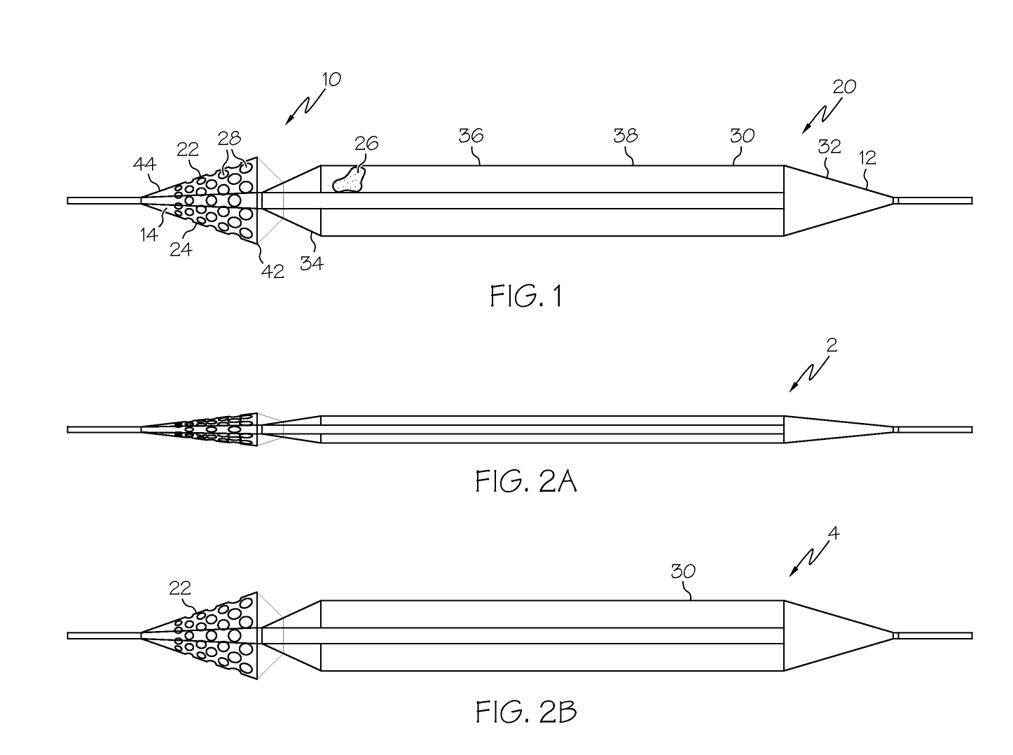

In some embodiments, for example as shown in FIG. 1, a balloon catheter 20 comprises a drug loss protection device 10. The drug loss protection device 10 has a proximal portion 12 and a distal portion 14. The drug loss protection device 10 has a particulate filter 22, which, in some embodiments is disposed adjacent to a balloon 30 along a distal portion 14 of the drug loss protection device 10. In some embodiments, the particulate filter 22 has a plurality of openings 28 which permit particulates smaller than a predetermined size to pass through the particulate filter 22. Particulates larger than the predetermined size are prevented from passing through the particulate filter 22.

In some embodiments, the particulate filter 22 comprises a mesh 24 having openings 28. The mesh 24 can comprise openings 28 of only of one size, or of various sizes. The size of the openings 28 is selected according to the size of particulates which are desired to pass through the openings. Stated differently, the size of the openings 28 is selected according to the size of particulates which are desirably filtered out. In some embodiments, the openings 28 are between 10 and 500 microns.

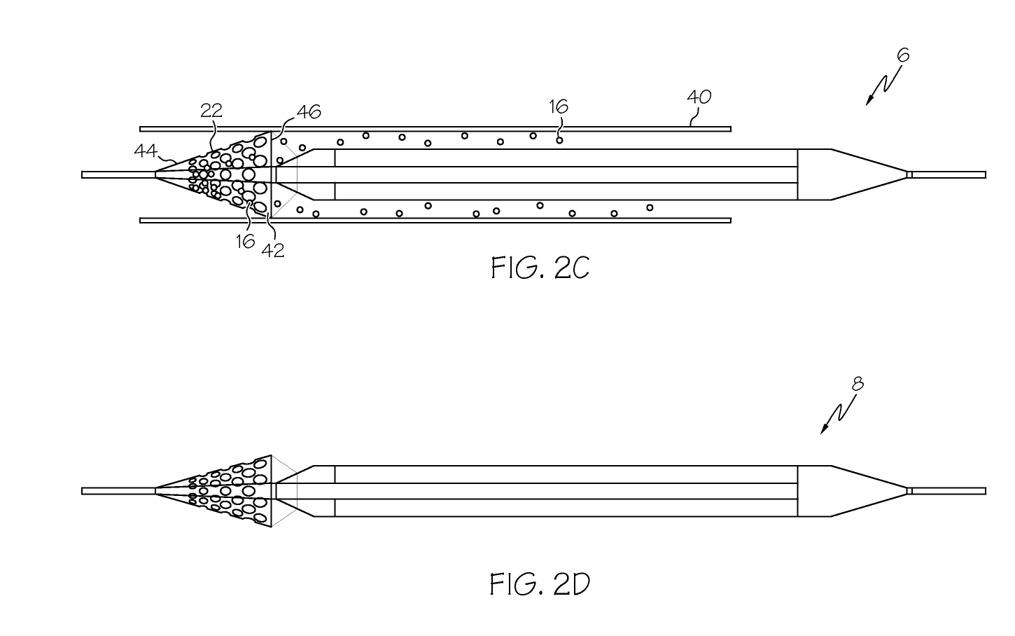

As shown in FIG. 1, the drug loss protection device 10 is in an expanded configuration. Additional configurations are shown in FIGS. 2A-2D. For example, the drug loss protection device 10 of FIG. 1 is shown in FIG. 2A in a delivery configuration 2. In FIG. 2B, the drug loss protection device 10 is shown in an expanded configuration 4. FIG. 2C shows the drug loss protection device 10 in a post-expansion configuration 6, and FIG. 2D shows the embodiment of FIG. 1 in an unexpanded configuration 8.

Returning to FIG. 1, a balloon 30 has proximal portion 32, a distal portion 34, and an intermediate portion 36 between the proximal and distal portions 32, 34. The balloon 30 can comprise any suitable configuration, for example as shown and described in US Publication No. 2007/0106216 to Noddin, which is herein incorporated by reference. In some embodiments, the balloon 30 has a drug 26 disposed on at least portion of a balloon outer surface 38.

In some embodiments, the balloon 30 comprises a drug coated balloon. As used herein, the term “drug coated balloon” is meant to include a balloon with a drug coating on the balloon, a balloon impregnated with a drug, a balloon having an excipient including a drug, a balloon having a polymer including a drug, or any other suitable balloon having a drug therein or thereon. Thus, in some embodiments, the drug 26 is applied directly to the balloon outer surface 38 or portion thereof. In some embodiments, the drug 26 is formulated with an excipient. An excipient is an additive to a drug-containing layer that facilitates adhesion to the balloon and/or release from the balloon upon expansion. The excipient may be a polymer, a contrast agent, a surface active agent, citrate ester, or other small molecule, examples of which are disclosed in U.S. Provisional Application No. 61/271,167 (Attorney Docket No. 563.2C-14586-US01) to Kangas et al. (“Nucleation of Drug Delivery Balloons to Provide Improved Crystal Size and Density”) and U.S. Pat. No. 6,409,816, which are herein incorporated by reference. In some embodiments, the drug 26 comprises a therapeutic agent. In some embodiments, the drug 26 can be applied to the balloon 30 as shown and described in US Publication No. 2007/0106216.

A therapeutic agent may be a drug or other pharmaceutical product such as non-genetic agents, genetic agents, cellular material, etc. Some examples of suitable non-genetic therapeutic agents include but are not limited to: anti-thrombogenic agents such as heparin, heparin derivatives, vascular cell growth promoters, growth factor inhibitors, Paclitaxel, etc. Where an agent includes a genetic therapeutic agent, such a genetic agent may include but is not limited to: DNA, RNA and their respective derivatives and/or components; hedgehog proteins, etc. Where a therapeutic agent includes cellular material, the cellular material may include but is not limited to: cells of human origin and/or non-human origin as well as their respective components and/or derivatives thereof. Where the therapeutic agent includes a polymer agent, the polymer agent may be a polystyrene-polyisobutylene-polystyrene triblock copolymer (SIBS), polyethylene oxide, poly vinyl propylene (PVP), silicone rubber and/or any other suitable substrate.

Turning to FIG. 2A, the balloon 30 and particulate filter 22 are shown in a delivery configuration 2. In the delivery configuration 2, the balloon 30 and particulate filter 22 have a reduced profile wherein the balloon 30 and the particulate filter 22 have a reduced cross-section to allow for intraluminal delivery of the device.

As shown in FIG. 2B, the balloon 30 and particulate filter 22 are shown in an expanded configuration 4. As shown in FIG. 2B, the balloon 30 and the particulate filter 22 are expanded to their maximum respective intraluminal profiles. The term “maximum intraluminal profile” refers to the largest possible cross-section which the component (e.g., balloon, filter) attains during deployment inside the body lumen. Thus, the “maximum intraluminal profile” may in fact be smaller than any actual maximum profile, for example if the device (or relevant component, e.g., balloon, filter) were expanded outside of the body lumen. To that end, the “maximum intraluminal profile” is the maximum profile attained during deployment of the device within a body lumen.

FIG. 2C shows the balloon 30 and particulate filter 22 in post-expansion configuration 6. In the post-expansion configuration 6, the profile of the balloon 30 is reduced from the expanded configuration 4 of FIG. 2B. The filter 22 has as large, or nearly as large, of a profile as in the expanded configuration 4. In this way, the filter 22 (or a portion thereof) remains in contact with the body lumen 40 even though the balloon 30 is reduced from its expanded configuration 4.

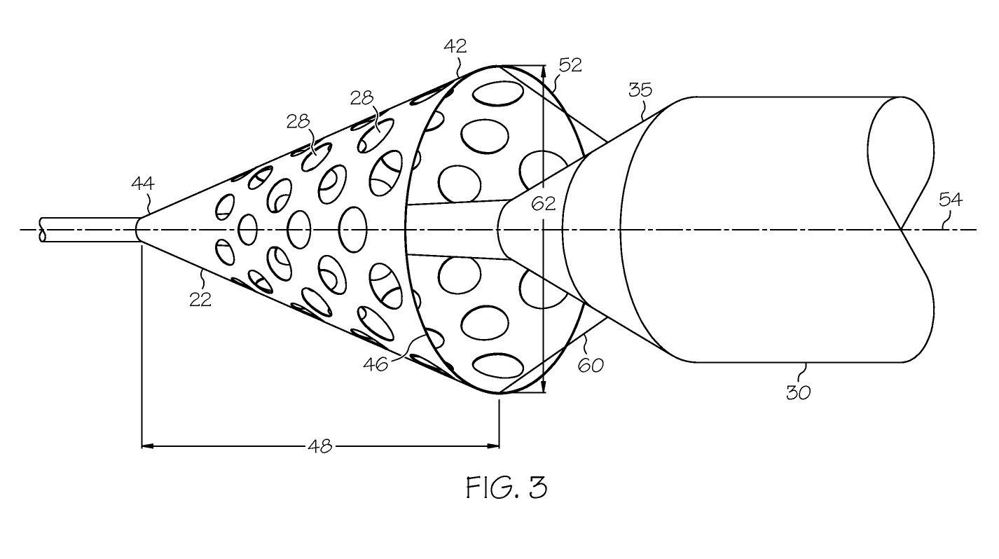

With further reference to FIG. 2C, in some embodiments, the filter 22 comprises a filter proximal portion 42 and a filter distal portion 44. In some embodiments, the filter 22 has a frusto-conical shape, for example as shown in FIG. 2C. In some embodiments, frusto-conical shaped filter 22 has a base 46 (FIG. 3), and a length 48 (FIG. 3). In some embodiments, the filter has a width 62. In some embodiments, the width 62 can also be referred to as the diameter of the filter, for example where the filter has a circular cross-section. In some embodiments, the base 46 is greater than the length 48 when the filter is in an expanded configuration 4 or post-expansion configuration 6. In some embodiments, the length 48 is greater than the width 62 of the base 46 when the filter is in an expanded configuration 4 or post-expansion configuration 6. The filter 22 can also comprise any suitable shape, for example, conical, conical with an elliptical cross-section, semi-spherical, cylindrical, or combinations thereof. In addition, where the filter 22 is conical or frusto-conical, it can be a right-cone, an oblique cone, or any other suitably shaped cone. In some embodiments, the base 46 is perpendicular to the longitudinal axis 54 of the balloon catheter 20 and/or body lumen 40. In some embodiments, the base 46 is arranged at an oblique angle relative to the longitudinal axis of the balloon catheter 20 and/or body lumen 40.

In some embodiments, the base 46 has a perimeter 52, for example as shown in FIG. 3. In the post-expansion configuration 6, the perimeter 52 can remain in contact with the body lumen 40 in order to prevent drug particulates 16 from bypassing the filter 22.

Turning again to FIG. 2D, the balloon 30 and particulate filter 22 are shown in an unexpanded configuration 8. When the balloon 30 and particulate filter 22 are in the unexpanded configuration 8, the balloon catheter 20 is able to be removed from the body lumen. In the unexpanded configuration 8, both the filter 22 and the balloon 30 have a smaller profile than when the drug loss protection device 10 is in its post-expansion configuration 6. Although, in some embodiments, neither the filter 22 nor the balloon 30 have as small a cross-sectional profile as when they are in the delivery configuration 2, their respective profiles nonetheless allow for extraction from the body lumen. Moreover, in some embodiments, the filter 22 will have drug particulates trapped in the filter 22 during removal of the balloon catheter 20.

With reference to FIG. 3, in some embodiments, the openings 28 of the filter 22 are larger in size near the filter proximal portion 42 than near the filter distal portion 44. In some embodiments, the openings 28 of the filter 22 are larger in size near the filter distal portion 44 than near the filter distal portion 42. In some embodiments, the openings 28 are all the same size. In some embodiments, the openings change in size in relation to the balloon inflation, deployment, and/or vessel size.

In some embodiments, the filter 22 of the drug loss protection device 10 is configured to expand in conjunction with the balloon 30. In some embodiments, at least a portion of the filter 22 is engaged to a distal cone 35.

In some embodiments, the drug loss protection device 10 comprises a plurality of interconnecting members 60. As shown in FIG. 3, the interconnecting members 60 extend from a portion of the filter 22, for example the perimeter 52. The interconnecting members 60 are further connected to a portion of the balloon 30, for example, the distal cone 35. In some embodiments, as the balloon 30 is expanded from a delivery configuration 2 (shown in FIG. 2A) to an expanded configuration 4 (FIG. 2B), the perimeter 52 is also expanded, along with the filter 22. The interconnecting members 60 can comprise any suitable metal, polymer, or other material. In some embodiments, the interconnecting members 60 comprise a shape memory material. In some embodiments, the interconnecting members 60 are self-expanding. In some embodiments, the interconnecting members 60 are balloon expandable. In some embodiments, the interconnecting members 60 comprise tethers.

Turning to FIG. 3B, in some embodiments, the drug loss protection device 10 comprises a single interconnecting member 60. In some embodiments, the interconnecting member is attached to a guidewire 50, filter wire, or other suitable device. In some embodiments, the filter 22 is attached as shown and described in U.S. Pat. No. 7,476,236, which is herein incorporated by reference. In some embodiments, the drug loss protection device 10 comprises a plurality of interconnecting members 60 attached to the balloon, guidewire 50, filter wire, or other suitable device. In some embodiments, the filter 22 is attached to both the balloon 30 and the guidewire or filter wire. In some embodiments, the filter 22 is permitted to rotate with respect to the balloon 30, guidewire 50, and/or filter wire.

In some embodiments, the interconnecting members 60 are attached to the balloon, filter, guidewire 50, and/or filter wire via an adhesive material. In some embodiments, the interconnecting members 60 are attached by laser weld. In some embodiments, the filter 22 is attached with a string or tether. In some embodiments, one or more of the interconnecting members 60 is temporarily attached. One or more of the interconnecting members can also be permanently attached. In some embodiments, the filter 22 is permitted to expand due to blood or fluid flow therethrough.

In some embodiments, the filter 22 can be expanded from a delivery configuration 2 to an expanded configuration 4 by reducing the length 48 of the filter 22. For example, where the filter 22 is frusto-conical in shape, the width 62 of the base 46 is increased as the length 48 of the filter 22 is decreased. In this way, the configuration of the filter 22 can be selected independently of the balloon configuration. Moreover, the filter 22 can remain in an expanded configuration even as the balloon 30 is deflated from an expanded configuration to a post-expansion configuration. In addition, the filer 22 can remain in a post-expansion configuration even where the balloon 30 is in an unexpanded configuration. Other intermediate configurations for both the filter 22 and balloon 30 are also possible, at least where the filter and balloon are capable of being independently configured.

In some embodiments, the length 48 of the filter 22 is adjusted by expansion of the filter 22. In some embodiments, the length 48 of the filter 22 is adjusted by a tether to the balloon 30, or guidewire, or the balloon 30 and the guidewire. In some embodiments, the entire filter 22 is permitted to translate distally or proximally relative to the balloon 30, guidewire 50, and/or filter wire, allowing the filter 22 to be placed in the desired location in the vessel or other body lumen. In some embodiments, the filter distal portion 44 is permitted to translate distally or proximally relative to the balloon 30, guidewire 50, and/or filter wire. In some embodiments, the filter 22, or a portion thereof can translate relative to the balloon 30 as the balloon 30 is inflated or deflated. In some embodiments, the balloon 30 inflation diameter controls the width 62 of the filter 22.

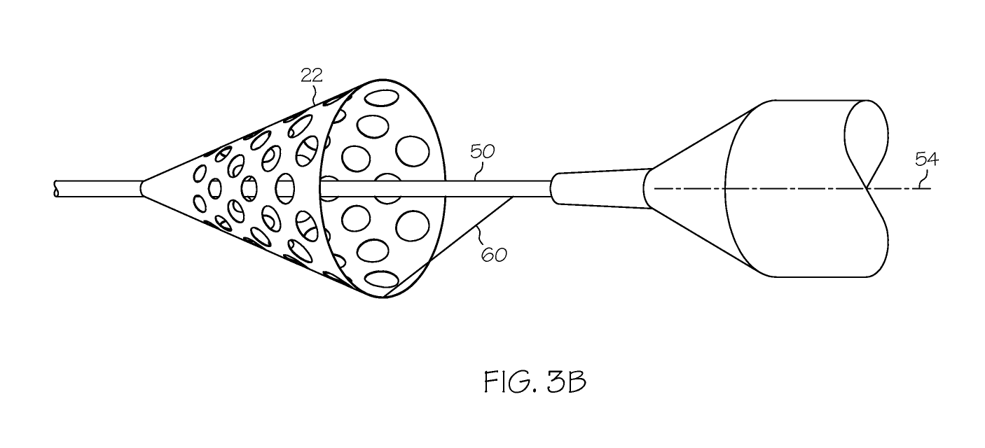

In some embodiments, for example as shown in FIG. 4, the filter 22 has one or more folds 64. In some embodiments, the folds 64 of the filter 22 will be present in the delivery configuration 2. The filter 22 can also comprise folds 64 in an unexpanded configuration 8 or in any other configuration.

In some embodiments, the filter 22 comprises an elastomeric material and is thus permitted to expand without folds. Suitable elastomeric materials include, nut are not limited to polyurethane, silicone, and rubber. In some embodiments, the elastomeric material is polyurethane having a durometer of between about 50 and 72. Other suitable elastomeric materials can also be used. In some embodiments, the filter 22 comprises both folds and an elastomeric material. In some embodiments, the filter 22 comprises non-elastic material. The filter 22 can also comprise shape memory metal or polymer. Examples of suitable filter materials include, but are not limited to Nitinol-block polymers, electro-active polymers (e.g., poly polypyrroles), and electro active metals (e.g., NiTi).

In some embodiments, for example as shown in FIG. 4, the balloon 30 has one or more folds 66. In some embodiments, the folds 66 will be present when the balloon catheter 20 is in the delivery configuration 2. The balloon 30 can also comprise folds 66 in an unexpanded configuration 8 or in any other configuration.

In some embodiments, the balloon 30 comprises an elastomeric material and/or one or more folds in conjunction therewith. The balloon can also comprise materials such as, but not limited to, those disclosed in U.S. Pat. No. 7,005,097, which is herein incorporated by reference.

In some embodiments, at least a portion of the filter 22 extends longitudinally over at least a portion of the balloon 30, for example as shown in FIG. 4. In some embodiments, at least a portion of the filter proximal portion 42 extends over at least a portion of the balloon distal portion 34. In some embodiments, the filter base 46 encircles at least a portion of the balloon 30, for example the balloon distal cone 35. In some embodiments, at least a portion of the filter 22 extends longitudinally over at least a portion of the balloon 30 in a delivery configuration 2. In some embodiments, at least a portion of the filter 22 extends longitudinally over at least a portion of the balloon 30 when either of the filter 22, the balloon 30, or both are in a delivery configuration. In some embodiments, at least a portion of the filter 22 extends longitudinally over at least a portion of the balloon 30 when either of the filter 22, the balloon 30, or both are in an expanded configuration. In some embodiments, at least a portion of the filter 22 extends longitudinally over at least a portion of the balloon 30 when either of the filter 22, the balloon 30, or both are in a post-expansion configuration. In some embodiments, at least a portion of the filter 22 extends longitudinally over at least a portion of the balloon 30 when either of the filter 22, the balloon 30, or both are in an unexpanded configuration. In some embodiments, the filter 22 is longitudinally offset from the balloon. In some embodiments, the filter does not overlap the filter.

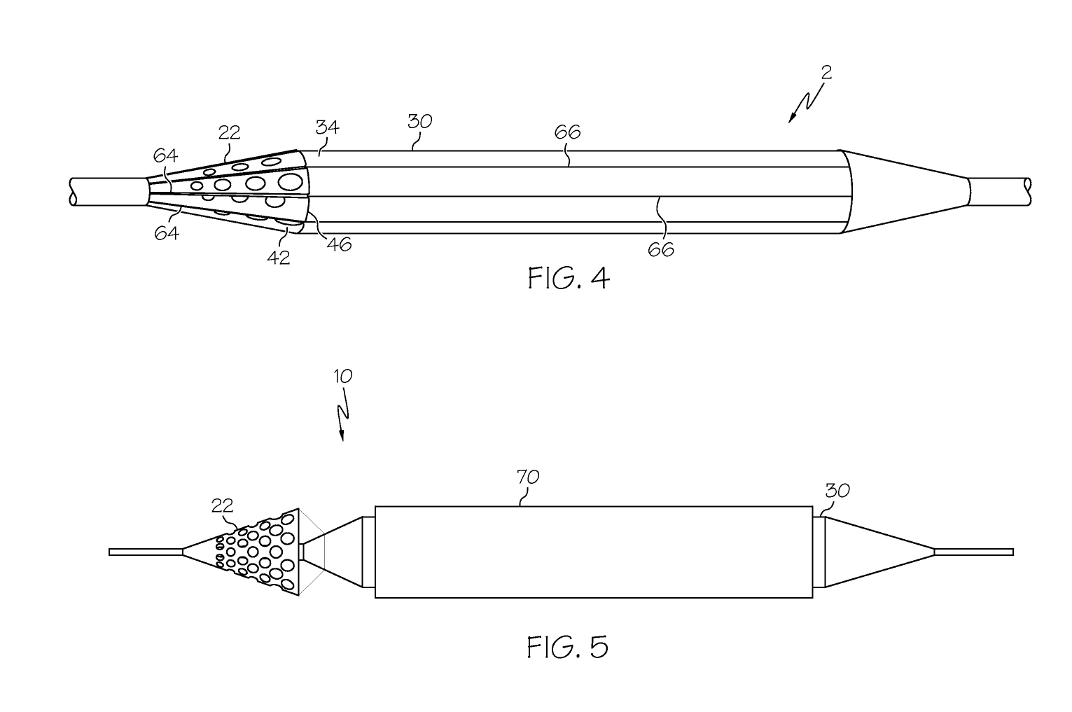

Turning now to FIG. 5, in at least one embodiment, the drug loss protection device 10 further comprises a stent 70 encircling at least a portion of the balloon 30. Suitable stents include, but are not limited to, those disclosed in U.S. Pat. No. 6,896,696 and US Publication Nos. 2002/0095208 and 2009/0240324, which are herein incorporated by reference.

In some embodiments, the stent 70 comprises a drug coated stent, a drug impregnated stent, a drug eluting stent, or any other suitable stent.

When used in conjunction with a drug eluting stent, the filter 22 of the drug loss protection device 10 can filter particulates that are emitted from the stent and/or balloon 30 during deployment of the stent 70 and/or expansion of the balloon 30.

In some embodiments, the stents are made from any suitable biocompatible materials including one or more polymers, one or more metals or combinations of polymer(s) and metal(s). Examples of suitable materials include biodegradable materials that are also biocompatible. By biodegradable is meant that a material will undergo breakdown or decomposition into harmless compounds as part of a normal biological process. Suitable biodegradable materials include polylactic acid, polyglycolic acid (PGA), poly(lactic-co-glycolic) acid (PLGA), collagen or other connective proteins or natural materials, polycaprolactone, hylauric acid, adhesive proteins, co-polymers of these materials as well as composites and combinations thereof and combinations of other biodegradable polymers. Other polymers that may be used include polyester and polycarbonate copolymers. Examples of suitable metals include, but are not limited to, stainless steel, titanium, tantalum, platinum, tungsten, gold and alloys of any of the above-mentioned metals. Examples of suitable alloys include platinum-iridium alloys, cobalt-chromium alloys including Elgiloy and Phynox, MP35N alloy and nickel-titanium alloys, for example, Nitinol.

In some embodiments, the stents are made of shape memory materials such as superelastic Nitinol or spring steel, or are made of materials which are plastically deformable. In the case of shape memory materials, in some embodiments, the stent is provided with a memorized or pre-set shape and then deformed to a reduced diameter shape. The stent may restore itself to its memorized or pre-set shape upon being heated to a transition temperature and having any restraints removed therefrom.

In some embodiments, the stents are created by methods including cutting or etching a design from a tubular stock, from a flat sheet which is cut or etched and which is subsequently rolled or from one or more interwoven wires or braids. Any other suitable technique which is known in the art or which is subsequently developed may also be used to manufacture the stents disclosed herein.

In some embodiments, the drug loss protection device 10 further comprises a stent-graft, graft, or any other suitable luminal scaffolding device.

In some embodiments, at least a portion of the drug loss prevention device is configured to include one or more mechanisms for the delivery of a therapeutic agent. In some embodiments, the stent will include one or more such mechanisms. Often the agent will be in the form of a coating or other layer (or layers) of material placed on a surface region of the stent or balloon, which is adapted to be released at the site of the stent's implantation or areas adjacent thereto.

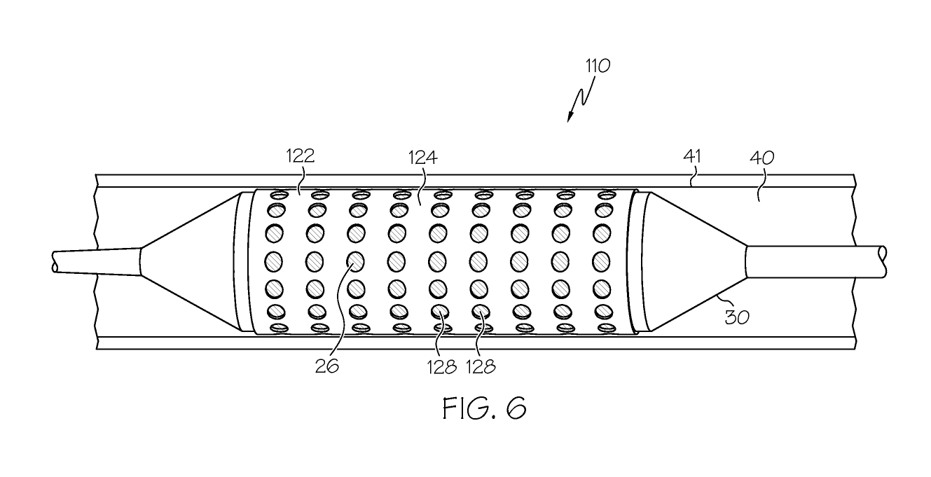

In at least one embodiment, for example as shown in FIG. 6, a drug loss protection device 110 comprises a balloon 30 and a filter 122. The filter 122 is adjacent to the balloon 30 and encircles at least a portion of the balloon 30. The balloon 30 can have a drug 26 disposed thereon or within a portion of the wall of the balloon. The filter 122 thereby surrounds the drug 26.

In some embodiments, the filter 122 comprises a mesh 124 which defines a plurality of openings 128. The size of the openings 128 is determined according to the size of drug particulates which are to pass through the openings 128, or conversely, according the size of drug particulates which are not to pass through the openings 128. Upon expansion of the balloon 30, the drug is pushed radially outwardly through the openings 128 and onto the adjacent luminal surface 41. Drug particulates larger than the openings 128 are not permitted to leave the filter 122, and thus do not exit the confines of the drug loss protection device 110. As such, particulates larger than the predetermined size are not permitted to freely enter a blood stream, for example where the drug loss protection device 110 is used in an artery or vein. In some embodiments, the openings change in size in relation to the balloon inflation, deployment, and/or vessel size.

After delivery of the drug 26, the balloon 30 of the drug loss prevention device 110 is deflated and the filter 122 and the balloon 30 assume a reduced profile. Drug particulates that were too large to pass through the openings 128 of the filter 122 are retained by the filter 122 and removed from the body lumen 40 along with the balloon catheter.

In some embodiments, the openings 28, 128 comprise pores, for example where the filter material is a porous polymeric material, expanded polytetrafluoroethylene, (ePTFE), or a hydrogel.

In some embodiments, any of the filters disclosed herein can also be used as an embolic filter.

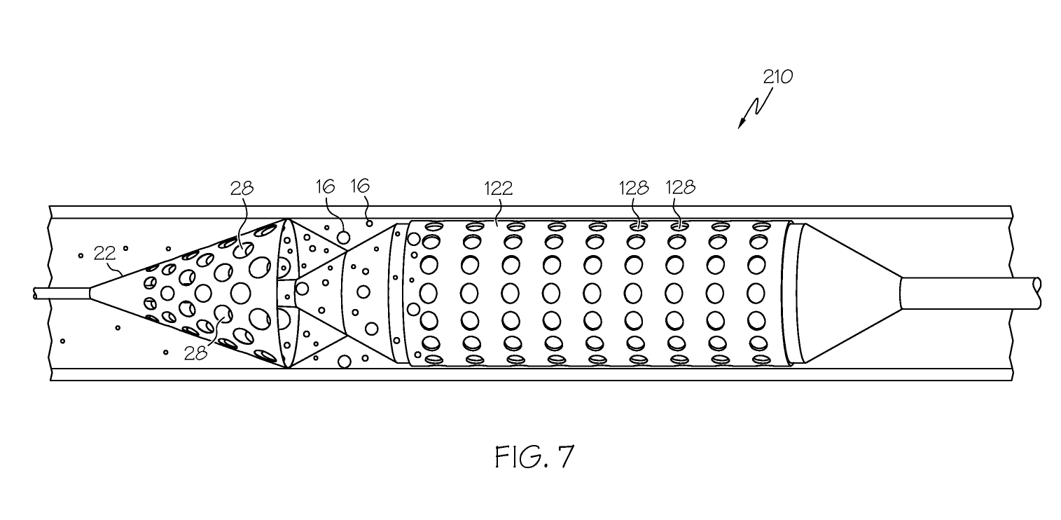

Turning to FIG. 7, in some embodiments, the drug loss protection device 210 comprises both a filter 22 and a filter 122. In some embodiments, the filter 122 has openings 128 that are larger than the openings 28 of the filter 22, for example where two stages of filters are desired. In some embodiments, the filter 122 has openings 128 that are smaller than the openings 28 of the filter 22, for example where filter 128 is configured to filter a first size of drug particulates 16 and filter 22 is configured to filter a second size of drug particulates. In some embodiments, the first size of drug particulates is larger than the second size of drug particulates. In some embodiments, the filter 122 is configured to filter out drug particulates and filter 22 is configured as an embolic filter. In this way, the drug protection device 210 has one filter which filters drug particulates and another filter which filters embolic material. Other suitable combinations and configurations of drug and embolic particulate filters are also contemplated. In some embodiments, the total number of filters can be more than one or two. In some embodiments, the number of filters can vary.

In some embodiments, the stent, the delivery system, the drug loss protection device, or other portion of the assembly includes one or more areas, bands, coatings, members, etc. that is (are) detectable by imaging modalities such as X-Ray, MRI, ultrasound, etc. In some embodiments at least a portion of the stent and/or adjacent assembly is at least partially radiopaque.

Description of some exemplary embodiments is contained in the following numbered paragraphs:

1. An intravascular drug loss protection device comprising:

a drug-coated balloon comprising a drug coating; and

a drug particulate filter distal to the drug-coated balloon, the drug particulate filter comprising a mesh, the mesh defining a plurality of openings configured to selectively prevent the passage of drug particulates therethrough.

2. The drug loss protection device of paragraph 1, wherein the drug-coated balloon and drug particulate filter each comprise a delivery configuration, an expanded configuration, a post-expansion configuration, and an unexpanded configuration;

the drug-coated balloon having a profile in each of the delivery configuration, expanded configuration, post-expansion configuration, and unexpanded configuration;

the particulate filter having a profile in each of the delivery configuration, expanded configuration, post-expansion configuration, and unexpanded configuration;

the profile of the drug-coated balloon in the expanded configuration being larger than in each of the delivery configuration, post-expansion configuration, and the unexpanded configuration;

the profile of the particulate in the expanded configuration being the same as in the post-expansion configuration.

3. The drug loss protection device of paragraph 1, wherein the drug particulate filter has a proximal end and a distal end, the drug particulate filter tapering from the proximal end to the distal end.
4. The drug loss protection device of paragraph 1, wherein the plurality of openings comprises openings of various sizes.
5. The drug loss protection device of paragraph 1, wherein the plurality of openings comprises openings that are all the same size.
6. The drug loss protection device of paragraph 1, wherein the openings are between 10 and 500 microns in size.
7. The drug loss protection device of paragraph 1 further comprising a stent encircling at least a portion of the drug-coated balloon.
8. The drug loss protection device of paragraph 7, wherein the stent comprises a drug eluting stent.
9. The drug loss protection device of paragraph 1 wherein the filter has a shape consisting of: semi-spherical, conical, frusto-conical, cylindrical, semi-cylindrical, and combinations thereof
10. An intravascular drug loss protection device comprising:

a drug-coated balloon comprising a drug coating;

a stent encircling at least a portion of the drug-coated balloon and engaged thereto; and

a drug particulate filter longitudinally adjacent to the drug-coated balloon and stent, the drug particulate filter defining a plurality of openings configured to selectively capture drug particulates.

11. The drug loss protection device of paragraph 10, wherein the filter is attached to a portion of the drug-coated balloon.
12. The drug loss protection device of paragraph 10, wherein the filter has a proximal portion, a distal portion, and a tapered profile tapering from the proximal portion to the distal portion.
13. The drug loss protection device of paragraph 12, wherein the filter is frusto-conical.
14. An intravascular drug loss protection device comprising:

a drug coated balloon comprising a drug coating; and

a drug particulate filter encircling at least a portion of the drug coated balloon; the drug particulate filter comprising a mesh, the mesh defining a plurality of openings sized to selectively prevent the passage of drug particulates therethrough.

15. The drug loss protection device of paragraph 14, wherein the openings are between 10 and 500 microns in size
16. A method for preventing drug loss into a body lumen comprising the steps of:

providing a drug-coated balloon comprising a drug coating;

providing a drug particulate filter adjacent to the drug-coated balloon;

expanding the drug particulate filter;

expanding the drug-coated balloon;

releasing drug particulates;

selectively capturing drug particulates in the drug particulate filter;

at least partially collapsing the drug particulate filter and balloon;

removing the drug-coated balloon and drug particulate filter from the body lumen.

17. The method of paragraph 16, wherein the step of releasing drug particulates comprises releasing drug particulates from the drug-coated balloon.
18. The method of paragraph 16 further comprising the step of providing a stent disposed around at least a portion of the drug-coated balloon.
19. The method of paragraph 16, wherein the drug coating comprises a plurality of drugs.

The above disclosure is intended to be illustrative and not exhaustive. This description will suggest many variations and alternatives to one of ordinary skill in this art. The various elements shown in the individual figures and described above may be combined or modified for combination as desired. All these alternatives and variations are intended to be included within the scope of the claims where the term “comprising” means “including, but not limited to”.

Further, the particular features presented in the dependent claims can be combined with each other in other manners within the scope of the Application such that the scope should be recognized as also specifically directed to other embodiments having any other possible combination of the features of the dependent claims. For instance, for purposes of claim publication, any dependent claim which follows should be taken as alternatively written in a multiple dependent form from all prior claims which possess all antecedents referenced in such dependent claim if such multiple dependent format is an accepted format within the jurisdiction (e.g. each claim depending directly from claim 1 should be alternatively taken as depending from all previous claims). In jurisdictions where multiple dependent claim formats are restricted, the following dependent claims should each be also taken as alternatively written in each singly dependent claim format which creates a dependency from a prior antecedent-possessing claim other than the specific claim listed in such dependent claim below.

This completes the description. Those skilled in the art may recognize other equivalents to the specific embodiment described herein which equivalents are intended to be encompassed by the claims attached hereto.

What is claimed is:

1. An intravascular drug loss protection device comprising:
a drug-coated balloon comprising a drug coating; and
a drug particulate filter distal to the drug-coated balloon, the drug particulate filter comprising a mesh, the mesh defining a plurality of openings configured to selectively prevent the passage of drug particulates therethrough.

2. The drug loss protection device of claim 1, wherein the drug-coated balloon and drug particulate filter each comprise a delivery configuration, an expanded configuration, a post-expansion configuration, and an unexpanded configuration;
the drug-coated balloon having a profile in each of the delivery configuration, expanded configuration, post-expansion configuration, and unexpanded configuration;
the particulate filter having a profile in each of the delivery configuration, expanded configuration, post-expansion configuration, and unexpanded configuration;
the profile of the drug-coated balloon in the expanded configuration being larger than in each of the delivery configuration, post-expansion configuration, and the unexpanded configuration;
the profile of the particulate in the expanded configuration being the same as in the post-expansion configuration.

3. The drug loss protection device of claim 1, wherein the drug particulate filter has a proximal end and a distal end, the drug particulate filter tapering from the proximal end to the distal end.

4. The drug loss protection device of claim 1, wherein the plurality of openings comprises openings of various sizes.

5. The drug loss protection device of claim 1, wherein the plurality of openings comprises openings that are all the same size.

6. The drug loss protection device of claim 1, wherein the openings are between 10 and 500 microns in size.

7. The drug loss protection device of claim 1 further comprising a stent encircling at least a portion of the drug-coated balloon.

8. The drug loss protection device of claim 7, wherein the stent comprises a drug eluting stent.

9. The drug loss protection device of claim 1 wherein the filter has a shape consisting of: semi-spherical, conical, frusto-conical, cylindrical, semi-cylindrical, and combinations thereof.

10. An intravascular drug loss protection device comprising:
a drug-coated balloon comprising a drug coating;
a stent encircling at least a portion of the drug-coated balloon and engaged thereto; and
a drug particulate filter longitudinally adjacent to the drug-coated balloon and stent, the drug particulate filter defining a plurality of openings configured to selectively capture drug particulates.

11. The drug loss protection device of claim 10, wherein the filter is attached to a portion of the drug-coated balloon.

12. The drug loss protection device of claim 10, wherein the filter has a proximal portion, a distal portion, and a tapered profile tapering from the proximal portion to the distal portion.

13. The drug loss protection device of claim 12, wherein the filter is frusto-conical.

14. An intravascular drug loss protection device comprising:
a drug coated balloon comprising a drug coating; and
a drug particulate filter encircling at least a portion of the drug coated balloon; the drug particulate filter comprising a mesh, the mesh defining a plurality of openings sized to selectively prevent the passage of drug particulates therethrough.

15. The drug loss protection device of claim 14, wherein the openings are between 10 and 500 microns in size.
